(19) United States
(12) Patent Application Publication
(10) Pub. No.: US 2024/0158335 A1
(43) Pub. Date: May 16, 2024

(54) A PROCESS FOR THE PREPARATION OF POLYFLUOROALKYLAMINES FROM POLYFLUOROALKYLALCOHOLS

(71) Applicants: Bayer Aktiengesellschaft, Leverkusen (DE); Centre National de la Recherche Scientifique (CNRS), Paris (FR)

(72) Inventors: Sergii PAZENOK, Leichlingen (DE); David BERNIER, Lyon (FR); Frédéric LEROUX, Herrlisheim (FR); Laura SANTOS, Horbourg-Wihr (FR); Armen PANOSSIAN, Strasbourg (FR); Morgan DONNARD, Marlenheim (FR)

(21) Appl. No.: 18/546,348

(22) PCT Filed: Feb. 8, 2022
(86) PCT No.: PCT/EP2022/052968

§ 371 Date: Aug. 14, 2023

(30) Foreign Application Priority Data
Feb. 17, 2021 (EP) 21290008.8
Feb. 17, 2021 (EP) 212900088

(51) Int. Cl. C07C 209/62; C07D 209/48

(52) U.S. Cl. CPC C07C 209/62; C07D 209/48

(57) ABSTRACT

A process for the preparation of polyfluoroalkylamines wherein polyfluoroalkylalcohols are reacted with an imide in the presence of SO₂F₂ and an acid scavenger in a first step and the compound obtained is then reacted with an acid, base or hydrazine in a second step.

The present invention relates to a process for the preparation of polyfluoroalkylamine starting from polyfluoroalkyalcohols using Gabriel synthesis.

Polyfluoroalkylamines are important intermediates in the preparation of active substances. For instance, 2,2-difluoroethylamine can be used as an intermediate in the preparation of flupyradifurone.

Various methods for the preparation of fluoroalkylamines are known such as (a) method of reacting the corresponding polyfluoroalkylhalide with ammonia (e.g. Dickey et al., Industrial and Engineering Chemistry, 1956, No. 2, 209-213, US2002/0183557) or the corresponding alcohol with ammonia (JP2005002031A) (b) method of hydrogenating the corresponding nitrile or azide compound (U.S. Pat. No. 3,532,755, Mecinovic et al, Green Chem., 2018, 20, 4418-4442) (c) method of reduction of the corresponding polyfluoroalkylamide (e.g. Douglas et al., Chem. Commun., 2016, 52, 12195-12198 for CF₃CH₂NH₂, Husted & Ahlbrecht J. Am. Chem. Soc. 1953, 75, 7, 1605-1608 for CHF₂CH₂NH₂, Soloshonok et al., Tetrahedron Letters, 2002, 43, 5449-5452, for R_{F}CH₂NH₂with R_{F}=—CF₃, —C₂F₅, —C₃F₉, Papanastassiou & Bruni, J. Org. Chem. 1964, 29, 10, 2870-2872 for FCH₂CH₂NH₂).

In addition, WO-A-2012/101044 discloses a process for the preparation of 2,2-difluoroethylamine wherein 2,2-difluoro-1-chloroethane is reacted with an imide in the presence of an acid scavenger such as a base to obtain 2,2-difluoroethylamine.

WO-A-2011/012243 and WO-A-2012/095403 disclose a process for the preparation of 2,2-difluoroethylamine wherein 2,2-difluoro-1-chloroethane is reacted with ammonia to obtain 2,2-difluoroethylamine.

WO-A-2011/042376 discloses a process for the preparation of 2,2-difluoroethylamine wherein 2,2-difluoro-1-nitroethane is hydrogenated in the presence of a catalyst to obtain 2,2-difluoroethylamine.

WO-A-2011/069994 discloses a process for the preparation of 2,2-difluoroethylamine wherein difluoroacetonitril is catalytic hydrogenated and the difluoroethylamide thereby obtained is subsequently converted into 2,2-difluoroethylamine by adding an acid which is suitable for cleaving the difluoroethylamide.

WO-A-2012/062702 discloses a process for the preparation of 2,2-difluoroethylamine wherein 2,2-difluoro-1-chloroethane is reacted with a benzylamine compound and the N-benzyl-2,2-difluoroethaneamine compound thereby obtained is catalytic hydrogenated to obtain 2,2-difluoroethylamine.

WO-A-2012/062703 discloses a process for the preparation of 2,2-difluoroethylamine wherein 2,2-difluoro-1-chloroethane is reacted with prop-2-en-1-amine and subsequent removal of the allyl group (deallylation) from the N-(2,2-difluoroethyl)prop-2-en-1-amine thereby obtained.

The known processes are disadvantageous since they either have a very long reaction time with high temperature and high pressure with only a low yield, have expensive reagents or equipment or because the reaction mixtures are highly corrosive, for which reason the known processes are unsuitable for commercial-scale use.

US 2012/0190867 (WO-A-2012/101044) describes the utilization of HCF2CH2Cl (Freon 142) using a Gabriel synthesis. HCF2CH2Cl is environmental unfriendly, belongs to the class of ozone depleting substances (ODS) and its utilization is strictly limited. The reaction time in the described process is short, but, a high temperature (90 to 140° C.) is needed. In addition, the process might require the use of a catalyst.

M. Epifanov et al. describes in JACS 2018, 140, 16464-16468 a process for the SO₂F₂-mediated alkylation of primary and secondary amines with polyfluoroalcohols. In the examples given in the publication, only amines are presented which are linked to one or to two alkyl chains alkyl-NH₂ or R₂NH such as cyclohexylamine, morpholine, phenylalanine, N-methylbenzylamine etc. These amines show a high nucleophilicity and basicity (pKb 3.5-4.5) and have so far been successfully alkylated with low-reactive polyfluoroalcohols.

The authors (JACS, p. 16466), however, have also found that substrates with steric bulk alpha to the amine such as cyclohexylamine but also anilines are only poor substrates for this reaction and cannot be alkylated with polyfluoroalcohols with high reaction rates. Like other amines, aniline is a base (pKb=9.42) and nucleophilic, although it is a weaker base and a worse nucleophile than structurally similar aliphatic amines.

In the present invention, phthalimide is used which has two carbonyl groups alpha to the amine group in a ring system and can therefore also be considered to be a bulky substrate. It is also known that due to the electron-withdrawing (—M) effect of the two carbonyl groups, phthalimide has a pronounced NH-acidity and no basicity at all. The high acidity of the imido-NH is the result of the pair of flanking electrophilic carbonyl groups. In addition, it is generally known that amides (like phthalimide or succinimide which are used in the process according to the invention) are generally less reactive than the amines (like those used in the process of Epifanov et al.) towards electrophiles.

It is therefore surprising that the polyfluoroalkylation of phthalimide (which is acidic and not basic) in the process according to the invention can be performed with high yields under mild conditions where at the same time cyclohexylamine or aniline are only poor substrates for this reaction. The same applies when succinimide is used instead of phthalimide.

The synthesis of polyfluoroalkyl amines from N-polyfluoroalkyl phtalimides was described by Kuwabara et al. in “The journal of the chemical society of Japan, 1985 v. 1985, N 4, p.796-798 (R_{F}CH₂NH₂with R_{F}=—CF₃, —CF₂CHF₂, (CF₂CF₂)₂H, —(CF₂CF₂)₃H). The preparation of the desired N-polyfluoroalkyl phtalimides from polyfluoroalkyl o-nitrobenzenesulfonates and K-salt of phtalimides was achieved under very harsh reaction conditions, prolonged heating at 150° C.

Starting from the known processes for the preparation of polyfluoroalkylamine (including 2,2-difluoroethylamine), the question now arises of how polyfluoroalkylamine including 2,2-difluoroethylamine can be prepared in a simple and inexpensive way from commercially available and environmentally friendly starting material such as polyfluoroalkylalcohols and cheap SO₂F₂ gas. The inventors have found that polyfluoroalkylamine can be prepared particularly advantageously from polyfluorinated alkylalcohols if an imide intermediate is first prepared and then cleaved.

A subject-matter of the invention is accordingly a process for the preparation of polyfluoroalkylamines of formula (IV)

R_{F}CH₂NH₂ (IV)
in which R_{F} is defined as in step (i) below comprising the following steps:

Step (i): Reaction of polyfluoroalkylalcohols of the formula (I)

R_{F}CH₂OH (I)
in which R_{F}=CHF₂, CF₃, C₂F₅ or HCF₂CF₂,
with an imide of the formula (II)

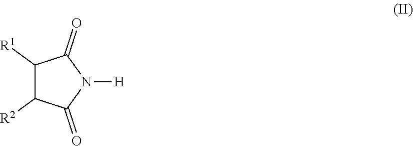

in the presence of SO₂F₂ and an acid scavenger to give a compound of the formula (III)

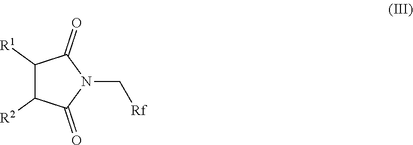

in which, in the compounds of the formulae (II) and (III), R¹ and R² are, each independently of one another, hydrogen or C₁-C₆-alkyl or R¹ and R² form, together with the carbon atoms to which they are bonded, a six-membered aromatic ring which is optionally substituted with halogen or C₁-C₁₂-alkyl;

Step (ii): Reaction of the compound of the formula (III) with an acid, base or hydrazine (i.e. cleaving the compound of formula (III) by adding an acid, base or hydrazine).

In a preferred embodiment of the invention the polyfluoroalkylalcohol of the formula (I) is CHF₂CH₂OH and the polyfluoroalkylamine of formula (IV) is CHF₂CH₂NH₂ (2,2-difluoroethyl-1-amine).

The imide of the formula (II) used in step (i) can also be present as salt. Such salts are in some cases commercially available (e.g., potassium salt of phthalimide). Before the salt is used in the process according to the invention, the imide of the formula (II) can also be converted to a salt by reaction with a suitable base. Suitable bases are known to a person skilled in the art or comprise the bases mentioned in the present case as acid scavenger.

It is preferable, in the process according to the invention, to use a compound of the formula (II) in which R¹ and R² are each hydrogen (i.e., succinimide) or in which R¹ and R² form, together with the carbon atoms to which they are bonded, a six-membered aromatic ring (i.e., phthalimide). If succinimide is used as compound of the formula (II), the compound of the formula (III-a) is obtained in step (i). If phthalimide is used as compound of the formula (II), the compound of the formula (III-b) is obtained in step (i):

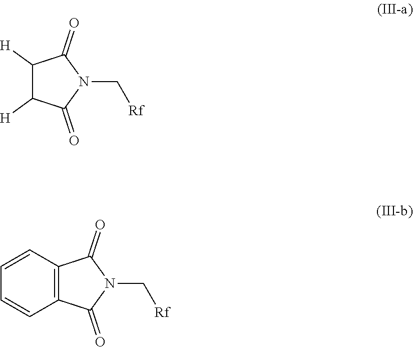

The process according to the invention can be illustrated by the following scheme:

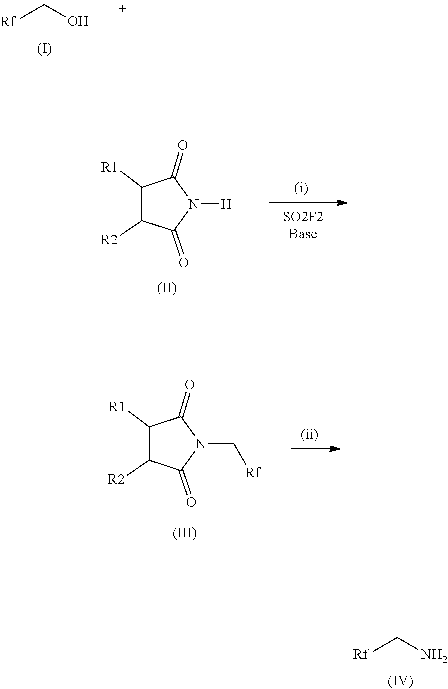

The utilization of SO₂F₂ for the N-alkylation of amines is known. Secondary or tertiary polyfluoroalkylamines can be prepared from polyfluoroalkylalcohols R_{F}CH₂OH with R_{F}=CF₃, CHF₂, CF₂CF₃, CF₂CF₂CF₃ according to Epifanov et al. in “JACS, 2018, 140, 16464-16468”. Cyclic tertiary amines can be isolated with a maximum yield of 67% (e.g. morpholine). Phtalimides can react easily with various non-fluorinated aliphatic alcohols in Sammis et al. Chem. Eur. J. 2020, 4958-4962. Intuitively, the inventors described the utilization of SO₂F₂ for the preparation of primary polyfluoralkyloamines through the synthesis of phtalimides.

It was likewise surprising that the polyfluorinated alcohols used in step (i) can be converted very well and with a high yield of about 85-90% to the imide of the formula (III).

Compounds of the formula (I) and (II) are known, are commercially available or can be prepared according to normal methods. SO₂F₂ is commercially available being used as an insecticide.

Unless otherwise indicated, the expression “alkyl”, in isolation or in combination with other terms, refers to linear or branched saturated hydrocarbon chains with up to 12 carbon atoms, i.e. C₁-C₁₂-alkyl, preferably with up to 6 carbon atoms, i.e. C₁-C₆-alkyl, very preferably with up to 4 carbon atoms, i.e. C₁-C₄-alkyl. Examples of such alkyls are methyl, ethyl, n-propyl, isopropyl, n-butyl, isobutyl, s-butyl, t-butyl, n-pentyl, n-hexyl, n-heptyl, n-octyl, n-nonyl, n-decyl, n-undecyl and n-dodecyl. The alkyls can be substituted with suitable substituents, e.g. with halogen.

Unless otherwise indicated, the expression “aryl” or “six-membered aromatic ring” refers to a phenyl ring.

Unless otherwise indicated, “halogen” or “hal” is fluorine, chlorine, bromine or iodine.

The reaction of alcohols of the formula (I) with an imide of the formula (II) in step (i) usually carried out in the presence of a solvent.

In the event that a solvent is added to the reaction mixture in step (i), it is preferably used in such an amount that the reaction mixture remains satisfactorily stirrable during the entire process. Use is advantageously made, based on the volume of the alcohols used, of the solvent in an amount of 1 to 50 times, preferably in an amount of 2 to 40 times and particularly preferably in an amount of 2 to 20 times. The term “solvent” is also understood to mean, according to the invention, mixtures of pure solvents.

All organic solvents which are inert under the reaction conditions are suitable solvents. Suitable solvents according to the invention are in particular ethers (e.g., ethyl propyl ether, methyl tert-butyl ether, n-butyl ether, anisole, phenetole, cyclohexyl methyl ether, dimethyl ether, diethyl ether, dimethyl glycol, diphenyl ether, dipropyl ether, diisopropyl ether, di-n-butyl ether, diisobutyl ether, diisoamyl ether, ethylene glycol dimethyl ether, isopropyl ethyl ether, diethylene glycol dimethyl ether, triethylene glycol dimethyl ether, tetrahydrofuran, 2-methyltetrahydrofuran, dioxane, and ethylene oxide and/or propylene oxide polyethers); compounds such as tetrahydrothiophene dioxide and dimethyl sulphoxide, tetramethylene sulphoxide, dipropyl sulphoxide, benzyl methyl sulphoxide, diisobutyl sulphoxide, dibutyl sulphoxide or diisoamyl sulphoxide; sulphones, such as dimethyl, diethyl, dipropyl, dibutyl, diphenyl, dihexyl, methyl ethyl, ethyl propyl, ethyl isobutyl and tetramethylene sulphone; aliphatic, cycloaliphatic or aromatic hydrocarbons (e.g., pentane, hexane, heptane, octane, nonane, such as white spirits with components with boiling points in the range, for example, from 40° C. to 250° C., cymene, benzine fractions within a boiling point interval from 70° C. to 190° C., cyclohexane, methylcyclohexane, petroleum ether, ligroin, octane, benzene, toluene or xylene); halogenated aromatic compounds (e.g., chlorobenzene or dichlorobenzene); amides (e.g., hexamethylphosphoramide, formamide, N,N-dimethylacetamide, N-methylformamide, N,N-dimethylformamide, N,N-dipropylformamide, N,N-dibutylformamide, N-methylpyrrolidine, N-methylcaprolactam, 1,3-dimethyl-3,4,5,6-tetrahydro-2(1H)-pyrimidine, octylpyrrolidone, octylcaprolactam, 1,3-dimethyl-2-imidazolinedione, N-formylpiperidine or N,N′-1,4-diformylpiperazine); nitriles (e.g., acetonitrile, propionitrile, n-butyronitrile, isobutyronitrile or benzonitrile); ketones (e.g., acetone) or mixtures thereof.

Acetonitrile, Dichloromethane, N,N-Dimethylformamide, N,N-dimethylacetamide, tetramethylene sulphone, N-methylpyrrolidone are preferred solvents in step (i).

The reaction of step (i) is carried out in the presence of one or more acid scavengers which are able to bind the hydrogen fluoride released in the reaction. In a preferred embodiment of the invention the acid scavenger used in step (i) is a base.

Organic and inorganic bases which are able to bind the hydrogen fluoride released are suitable acid scavengers. Examples of organic bases are tertiary nitrogen bases, such as, e.g., tertiary amines, substituted or unsubstituted pyridines and substituted or unsubstituted quinolines, triethylamine, trimethylamine, diisopropylethylamine, tri-n-propylamine, tri-n-butylamine, tri-n-hexylamine, tricyclohexylamine, N-methylcyclohexylamine, N-methylpyrrolidine, N-methylpiperidine, N-ethylpiperidine, N,N-dimethylaniline, N-methylmorpholine, pyridine, 2-, 3- or 4-picoline, 2-methyl-5-ethylpyridine, 2,6-lutidine, 2,4,6-collidine, 4-dimethylaminopyridine, quinoline, quinaldine, N,N,N,N-tetramethyl-ethylenediamine, N,N-dimethyl-1,4-diazacyclohexane, N,N-diethyl-1,4-diazacyclohexane, 1,8-bis(di-methylamino)naphthalene, diazabicyclooctane (DABCO), diazabicyclononane (DBN), diazabicycloundecane (DBU), butylimidazole and methylimidazole.

Examples of inorganic bases are alkali metal or alkaline earth metal hydroxides, hydrogencarbonates or carbonates and other inorganic aqueous bases; preference is given, e.g., to sodium hydroxide, potassium hydroxide, lithium hydroxide, calcium hydroxide, sodium carbonate, potassium carbonate, sodium hydrogencarbonate, potassium hydrogencarbonate and sodium acetate, KF, CsF. Potassium carbonate or sodium carbonate, KF and CsF are very particularly preferred.

The molar ratio of acid scavenger, in particular of abovementioned bases, to the imide of the formula (II) used usually lies in the range of from 1:1 to 5:1, preferably in the range of from 1:1 to 4:1 and particularly preferably in the range of from 1:1 to 3:1. The use of larger amounts of base is technically possible but is not useful economically.

The molar ratio of polyfluoralkylalcohols of the formula (I) to the imide of the formula (II) used, normally lies in the range of from 1:1 to 5:1, preferably in the range of from 1:1 to 3:1 and particularly preferably in the range of from 1:1 to 2,5:1.

The molar ratio of SO₂F₂ to the imide of the formula (II) used normally lies in the range of from 1:1 to 5:1, preferably in the range of from 1:1 to 3:1 and particularly preferably in the range of from 1:1 to 2:1.

The reaction of step (i) is carried out, in principle, in an open system or under intrinsic pressure in a pressure-vessel (autoclave). The pressure during the reaction (i.e., the intrinsic pressure) depends on the reaction temperature used, on the amount of SO₂F₂ and on the solvent used, if a solvent is present in step (i). If an increase in pressure is desired, an additional increase in pressure can be achieved by adding an inert gas, such as nitrogen or argon.

The most preferred operation modus is a bubbling of SO₂F₂ into the reaction mixture containing phtalimid, the base and the polyfluoroalkylalcohol of formula (I).

The process according to the invention can be carried out continuously or batchwise. It is likewise conceivable to carry out some steps of the process according to the invention continuously and the remaining steps batchwise. Continuous steps within the meaning of the invention are those in which the inflow of compounds (starting materials) into a reactor and the outflow of compounds (products) from the reactor take place simultaneously but separately in space, while, with batchwise steps, the sequence inflow of compounds (starting materials), optionally chemical reaction, and outflow of compounds (products) take place one after another chronologically.

It is preferable, in carrying out reaction step (i), for the internal temperature to lie in the range from −5° C. to 50° C., particularly preferably in the range from 10° C. to 40° C.

The reaction time of the reaction in step (i) is short and lies in the range from 0.5 to 5 hours. A longer reaction time is possible but is not useful economically.

The reaction mixture from step (i) is worked up depending on the physical properties of the product. If phthalimide or a substituted phthalimide is used as compound of the formula (II), first the solvent is removed under vacuum. If succinimide is used as compound of the formula (II), then first the solids are filtered off. Following that, the “diluting” of the reaction mixture, i.e. addition of water in which salts may be dissolved, is normally carried out. The product can then be isolated by filtration or can be extracted from the aqueous phase using an organic solvent.

In step (ii), the cleaving of the compound of the formula (III) to give polyfluoroalkylamines or a salt thereof is carried out by the addition of acid, base or hydrazine (including hydrazine hydrate). Preferably, an acid or hydrazine is used in step (ii). Particular preferred is the use of hydrazine hydrate. The typical procedure for this step is given in US 2012/0190867 or in “The journal of the chemical society of Japan, 1985 v. 1985 N. 4 p.796-798.

The bases which can be used in step (ii) are known to a person skilled in the art or comprise the bases mentioned in the present case as acid scavenger. The acids used in step (ii) are organic or inorganic acids, inorganic acids being preferably used. Examples of such preferred inorganic acids according to the invention are hydrochloric acid, hydrobromic acid, sulphuric acid and phosphoric acid.

The cleaving of the compound of the formula (III) in step (ii) is carried out in a suitable solvent. Here also, the solvent is preferably used in such an amount that the reaction mixture remains stirrable during the whole of the process. Use is advantageously made, based on the compound of the formula (III) used, of the solvent in an amount of approximately 1 to 50 times (v/v), preferably in an amount of approximately 2 to 40 times and particularly preferably in an amount of 2 to 10 times.

All organic solvents which are inert under the reaction conditions are possible as solvent. The term “solvent” is also understood to mean, according to the invention, mixtures of pure solvents.

Suitable solvents according to the invention in step (ii) are in particular water, ethers (e.g., ethyl propyl ether, methyl tert-butyl ether, n-butyl ether, anisole, phenetole, cyclohexyl methyl ether, dimethyl ether, diethyl ether, dimethyl glycol, diphenyl ether, dipropyl ether, diisopropyl ether, di-n-butyl ether, diisobutyl ether, diisoamyl ether, ethylene glycol dimethyl ether, isopropyl ethyl ether, diethylene glycol dimethyl ether, triethylene glycol dimethyl ether, tetrahydrofuran, 2-methyltetrahydrofuran, dioxane, and ethylene oxide and/or propylene oxide polyethers); aliphatic, cycloaliphatic or aromatic hydrocarbons (e.g., pentane, hexane, heptane, octane, nonane, such as white spirits with components with boiling points in the range, for example, from 40° C. to 250° C., cymene, benzene fractions within a boiling point interval from 70° C. to 190° C., cyclohexane, methylcyclohexane, petroleum ether, ligroin, octane, benzene, toluene or xylene); linear and branched carboxylic acids (e.g., formic acid, acetic acid, propionic acid, butyric acid and isobutyric acid) and the esters thereof (e.g., ethyl acetate and butyl acetate); alcohols (e.g., methanol, ethanol, isopropanol, n-butanol and isobutanol) or mixtures thereof. Preferred solvents according to the invention in step (ii) are methanol, ethanol and water or mixtures thereof.

The molar ratio of acid or hydrazine (or hydrazine hydrate) to the compound of the formula (III) used lies in the range of from 0.8:1 to 10:1, preferably in the range of from 1:1 to 5:1 and particularly preferably in the range of from 1:1 to 3:1. The addition of larger amounts of acid or hydrazine is possible in principle. With suitable manageability, the acid can also be used as solvent. The hydrazine is used in the form of its hydrate.

The cleaving in step (ii) can be carried out at temperatures in the range of from 0° C. to 150° C. The internal temperature preferably lies in the range of from 20° C. to 100° C.; it particularly preferably lies in the range of from 40° C. to 70° C. For the cleavage with hydrazine the temperature preferably lies in the range of 50-70° C.

The reaction time for the cleaving is short and lies in the range from 0.1 to 12 hours. A longer reaction time is possible but is not useful economically.

After the end of the reaction, the polyfluoroalkylamines of formula (IV) obtained can be purified by distillation. Alternatively, the 2,2-difluoroethylamine can also be isolated and purified as salt, e.g. hydrochloride. The 2,2-difluoroethylamine salt can subsequently be released by addition of base, preferably NaOH.

In a most preferred embodiment of the invention the polyfluoroalkylalcohol of the formula (I) is CHF₂CH₂OH and the polyfluoroalkylamine of formula (IV) is 2,2-difluoroethyl-1-amine

Further in a most preferred embodiment of the invention the compound of formula (II) is phthalimide and the compound of formula (III) is the compound of formula (III-b).

Further in a most preferred embodiment of the invention in step (i) diazabicycloundecane is used as a base (acid scavenger).

Further in a most preferred embodiment of the invention in step (ii) hydrochloric acid is used.

Further in a most preferred embodiment of the invention in step (ii) hydrazine hydrate is used.

### PREPARATION EXAMPLES

### Example 1

### Preparation of 2-(2,2-difluoroethyl)-1H-isoindole-1,3(2H)-dione (step (i))

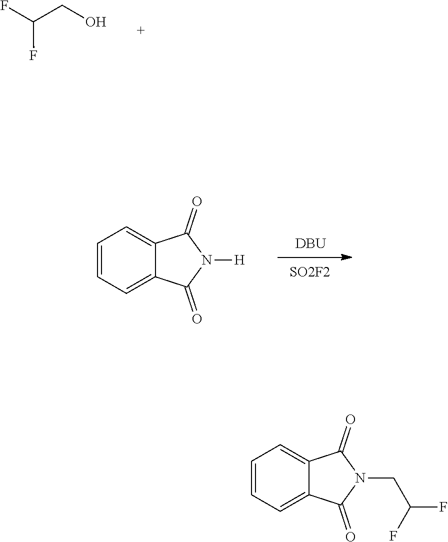

### Example 1.1

1.47 g (0.01 mmol) of phtalimide, 1.45 mL (0.02 mol) of 2,2-difluoroethanol and 6 g (0.04 mol) of diazabicycloundecane were placed in 25 mL of N,N-Dimethylacetamide. 2.2 g (0.02 mol) SO₂F₂ was slowly bubbled through the reaction mixture at 20 ° C. for 40 min. The solvent was removed under vacuum of 1 mbar. The concentrated solution was diluted with methyl tert-butyl ether and washed with water. The organic layer was collected, dried with magnesium sulfate and filtered. The removal of ether under vacuum yielded 1,97 g of a white solid with purity of 98%, yield 91%. M.p. 114-116° C.

¹H NMR (DMSO): 7.95-7-87 (m, 4H), 6.25 (tt, 1H), 4.0 (td, 2H) ppm.

¹³C NMR (DMSO): 167.37, 134.93, 131.51, 123.54, 113.54 (t), 39.70 (t) ppm.

¹⁹F NMR (DMSO): 121.40 (dt) ppm.

### Example 1.2

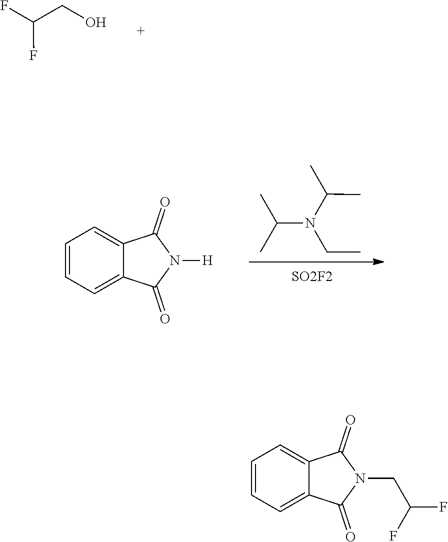

1.47 g (0.01 mmol) of phtalimide, 1.45 mL (0.02 mol) of 2,2-difluoroethanol and 3.88 g (0.03 mol) of N-ethyldiisopropylamine were placed in 25 mL of N,N-Dimethylacetamide. 3.06 g (0.03 mol) SO₂F₂ was slowly bubbled through the reaction mixture at 40° C. for 3 h and the reaction mixture was stirred under SO₂F₂ atmosphere for 5 h at 40° C. The solvent was removed under vacuum and reaction mixture diluted with water. The precipitate was filtered off and dried. Obtained 1.81 g of a white solid with purity of 100%, yield 86%. M.p. 114-116° C.

¹H NMR (DMSO): 7.95-7-87 (m, 4H), 6.25 (tt, 1H), 4.0 (td, 2H) ppm.

¹³C NMR (DMSO): 167.37, 134.93, 131.51, 123.54, 113.54 (t), 39.70 (t) ppm.

¹⁹F NMR (DMSO): 121.40 (dt) ppm.

### Example 1.3

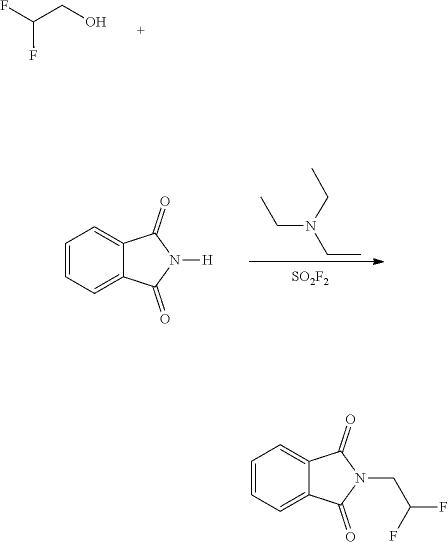

1.47 g (0.01 mmol) of phtalimide, 1.45 mL (0.02 mol) of 2,2-difluoroethanol and 3.1 g (0.03 mol) of triethylamine were placed in 25 mL of N,N-Dimethylacetamide. 3.06 g (0.03 mol) SO₂F₂ was slowly bubbled through the reaction mixture at 40° C. for 3 h and the reaction mixture was stirred under SO2F2 atmosphere for 12 h. at 40° C. The solvent was removed under vacuum and reaction mixture diluted with water. The precipitate was filtered off and dried. Obtained 1.9 g of a white solid with purity of 100%, yield 84%. M.p. 114-116° C.

¹NMR (DMSO): 7.95-7-87 (m, 4H), 6.25 (tt, 1H), 4.0 (td, 2H) ppm.

¹³C NMR (DMSO): 167.37, 134.93, 131.51, 123.54, 113.54 (t), 39.70 (t) ppm.

¹⁹F NMR (DMSO): 121.40 (dt) ppm.

### Example 2

### Preparation of 2-(2,2,2-trifluoroethyl)-1H-isoindole-1,3(2H)-dione (step (i))

### Example 2.1

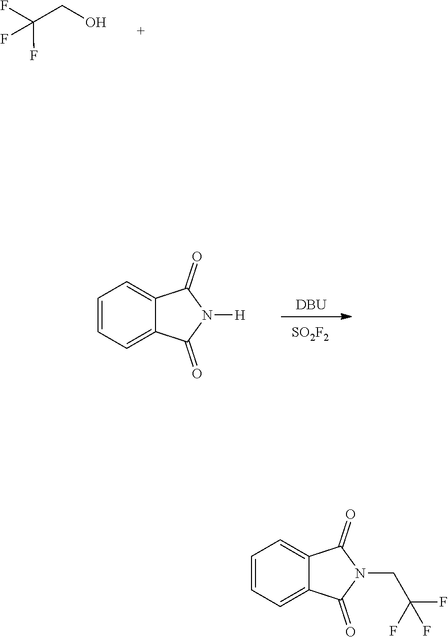

1.47 g (0.01 mol) of phtalimide, 1.8 mL (0.02 mol) of 2,2,2-trifluoroethanol and 4.5 g (0.03 mol) of diazabicycloundecane were placed in 25 mL of N,N-Dimethylacetamide. 2.2 g (0.02 mol) SO₂F₂ was bubbled through the reaction mixture at 20 ° C. for 60 min. The solvent was removed under vacuum and reaction mixture diluted with water. The precipitate was filtered off and dried. Obtained 2,1 g. of a white solid with purity of 100%, yield 92%. M.p. 122-127° C.

¹H NMR (DMSO): 7.99-7-90 (m, 4H), 4.43 (q, 2H) ppm.

¹³C NMR (DMSO): 166.86, 135.20, 131.30, 123.86 (q), 123.82, 38.89 (q) ppm.

¹⁹F NMR (DMSO): −68.85 (t, 3F) ppm.

Example 2.2

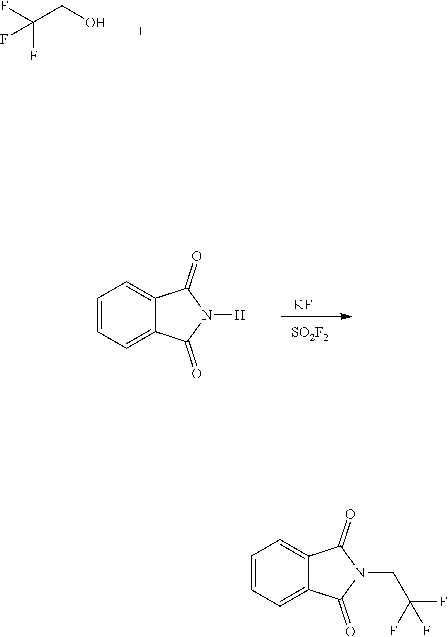

1.47 g (0.01 mol) of phtalimide, 1.8 mL (0.02 mol) of 2,2,2-trifluoroethanol and 2.3 g (0.04 mol) of spray-dried KF were placed in 25 mL of N,N-dimethylacetamide. 2.2 g (0.02 mol) SO₂F₂ was bubbled through the reaction mixture at 30° C. for 40 min and the reaction mixture was stirred under SO2F2 atmosphere for 12 h. The solvent was removed under vacuum and reaction mixture diluted with water. The precipitate was filtered off and dried. Obtained 1.98 g of white solid with purity of 100%, yield 86%. M.p. 122-127° C.

¹H NMR (DMSO): 7.99-7-90 (m, 4H), 4.43 (q, 2H) ppm.

¹³C NMR (DMSO): 166.86, 135.20, 131.30, 123.86 (q), 123.82, 38.89 (q) ppm.

¹⁹F NMR (DMSO): −68.85 (t, 3F) ppm.

### Example 3

### Preparation of 2-(2,2,3,3,3-pentafluoropropyl)-1H-isoindole-1,3(2H)-dione (step (i))

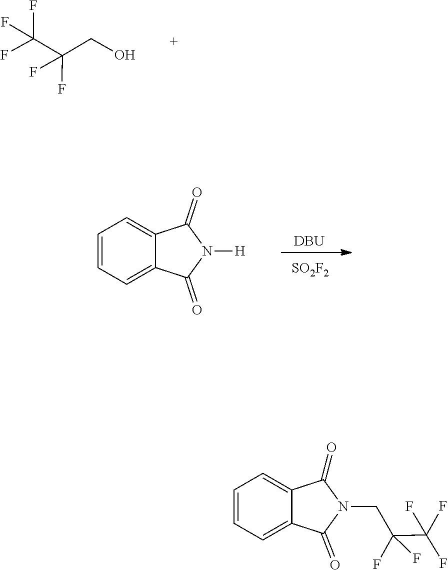

1.47 g (0.01 mol mmol) of phtalimide, 3 g (0.02 mol) of 2,2,3,3,3-pentafluopropanol and 4.5 g (0.03 mol) of diazabicycloundecane were placed in 25 mL of N,N-Dimethylacetamide. 2.55 g (0.025 mol) SO₂F₂ was bubbled through the reaction mixture at 20° C. for 60 min and the reaction mixture was stirred under SO2F2 atmosphere for 5 h. The solvent was removed under vacuum and the reaction mixture diluted with water. The precipitate was filtered off and dried. Obtained 2.53 g of a white solid with purity of 100%, yield 91%. M.p.134-135° C.

¹H NMR (DMSO): 8.00-7-90 (m, 4H), 4.42 (t, 2H) ppm.

¹³C NMR (DMSO): 166.92, 135.30, 131.25, 123.89, 118.40 (tq), 112.60 (m), 37.00 (t) ppm.

¹⁹F NMR (DMSO): −83.70 (s, 3F), −118.86 (t, 2F) ppm.

### Example 4

### Preparation of 2-(2,2,3,3-tetrafluoropropyl)-1H-isoindole-1,3(2H)-dione (step (i))

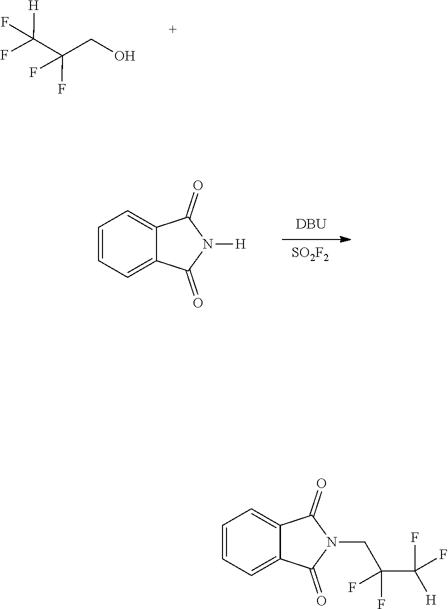

1.47 g (0.01 mol) of phtalimide, 3.3 g (0.02 mol) of 2,2,3,3-tetrafluopropanol and 4.5 g (0.03 mol) of diazabicycloundecane were placed in 25 mL of N,N-Dimethylacetamide. 2.55 g (0.025 mol) SO₂F₂ was bubbled through the reaction mixture at 20° C. for 60 min and the reaction mixture was stirred under SO2F2 atmosphere for 5 h. The solvent was removed under vacuum and the reaction mixture diluted with water. The precipitate was filtered off and dried. Obtained 2.3 g of a white solid with purity of 100%, yield 88%. M.p. 129-130° C.

¹H NMR (DMSO): 7.97-7-90 (m, 4H), 6.64 (tt, 1H), 4.23 (t, 2H) ppm.

¹³C NMR (DMSO): 167.22, 135.08, 131.50, 123.75, 114.98 (tt), 109.54 (tt), 37.43 (t) ppm.

¹⁹F NMR (DMSO): −120.95 (m, 2F), −138.64 (dt, 2F) ppm.

### Example 5

### Preparation of 2,2-difluoroethylamine (step (ii))

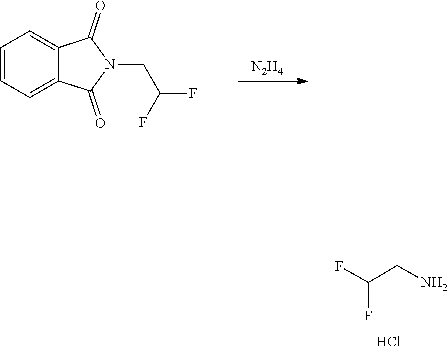 [SMILES: O=C1c2ccccc2C(=O)N1CC(F)F ; "N2H4" ; NCC(F)F ; Cl]

4.22 g (0.02 mol) of 2-(2,2-difluoroethyl)-1H-isoindole-1,3(2H)-dione was placed in 50 mL of ethanol and treated with 1.8 g (0.036 mol) of hydrazine hydrate. The reaction mixture was stirred 2 h at reflux. Subsequently, the reaction mixture was cooled to 20° C. and the solid was filtered off. The filtrate was adjusted to pH 2 with 10 mL of hydrochloric acid (2N) and concentrated to dryness to yield 2 g (85%) of hydrochloride salt of 2,2-difluoroethylamine.

¹⁹F NMR (DMSO): −122.10 (dt, 2F) ppm.

¹³C NMR (DMSO): 132.77, 125.32, 113.51 (t) ppm.

¹H NMR (DMSO): 6.39 (tt, 1H), 3.31 (m, 2H) ppm.

### Example 6

### Preparation of 2,2,3,3-tetrafluoropropan-1-amine (step (ii))

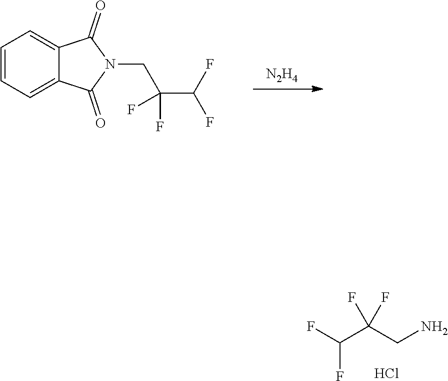

5.22 g (0.02 mol) of 2-(2,2,3,3-tetrafluoropropyl)-1H-isoindole-1,3(2H)-dione was placed in 50 mL of ethanol and treated with 1.4 g (0.028 mol) of hydrazine hydrate. The reaction mixture was stirred 2 h at reflux. Subsequently, the reaction mixture was cooled to 20° C. and the solid was filtered off. The filtrate was adjusted to pH 2 with 10 mL of hydrochloric acid (2N) and concentrated to dryness to yield 3.1 g yield 92% of hydrochloride salt of 2,2,3,3-tetrafluoropropan-1-amine.

¹⁹F NMR (DMSO): −121.08 (m, 2F), −137.84 (dt, 2F) ppm.

¹³C NMR (DMSO): 114.93 (tt), 109.24 (tt), 38.23 ppm.

¹H NMR (DMSO): 6.73 (tt, 1H), 3.62 (t, 2H) ppm.

### Example 7

### Preparation of 2,2,3,3,3-pentafluoropropan-1-amine (step (ii))

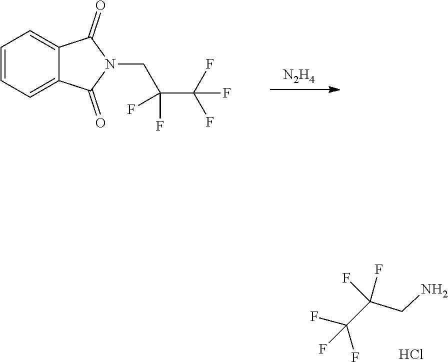 [SMILES: O=C1c2ccccc2C(=O)N1CC(F)(F)C(F)(F)F ; "N2H4" ; NCC(F)(F)C(F)(F)F ; Cl]

5.58 g (0.02 mol) of 2-(2,2,3,3,3-pentafluoropropyl)-1H-isoindole-1,3(2H)-dione was placed in 50 mL of ethanol and treated with 1.4 g (0.028 mol) of hydrazine hydrate. The reaction mixture was stirred 2 h at reflux. Subsequently, the reaction mixture was cooled to 20° C. and the solid was filtered off. The filtrate was adjusted to pH 2 with 10 mL of hydrochloric acid (2N) and concentrated to dryness to yield 3.37 g. (91%) of hydrochloride salt of 2,2,3,3,3-pentafluoropropan-1-amine.

¹⁹F NMR (DMSO): −83.37 (3F), −119.01 (t, 2F) ppm.

¹H NMR (DMSO): 3.91 (t, 2H) ppm.

### Example 8

### Preparation of 2,2,2-trifluoroethylamine (step (ii)

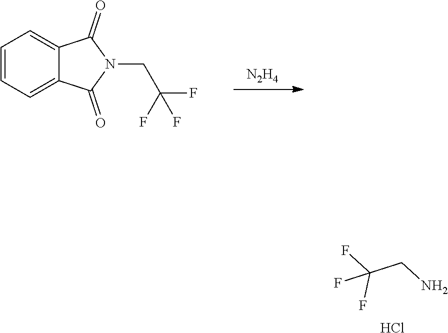

4.58 g (0.02 mmol) of 2-(2,2,2-trifluoroethyl)-1H-isoindole-1,3(2H)-dione was placed in 50 mL of ethanol and treated with 1.6 g (0.032 mol) of hydrazine hydrate. The reaction mixture was stirred 2 h at reflux. Subsequently, the reaction mixture was cooled to 20° C. and the solid was filtered off. The filtrate was adjusted to pH 2 with 10 mL of hydrochloric acid (2N) and concentrated to dryness to yield 2.45 g (90%) of hydrochloride salt of 2,2-difluoroethylamine.

¹⁹F NMR (DMSO): -67.89 (t, 3F)

¹H NMR (DMSO): 3.87 (q, 2H)

1. Process for preparation of a polyfluoroalkylamine of formula (IV)
R_{F}CH₂NH₂ (IV)
in which R_{F} is defined as in (i) below comprising:
(i): Reaction of polyfluoroalkylalcohols of formula (I)
R_{F}CH₂OH (I)
in which R_{F}=CHF₂, CF₃, C₂F₅ or HCF₂CF₂,
with an imide of formula (II) 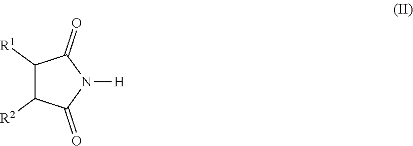
in the presence of SO₂F₂ and an acid scavenger to give a compound of formula (III) 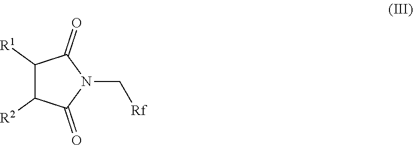
in which, in the compounds of formulae (II) and (III),
R¹ and R² are, each independently of one another, hydrogen or C₁-C₆-alkyl or R¹ and R² form, together with the carbon atoms to which they are bonded, a six-membered aromatic ring which is optionally substituted with halogen or C₁-C₁₂-alkyl;
(ii): Reaction of the compound of formula (III) with an acid, base or hydrazine.

2. The Process according to claim 1, wherein the polyfluoroalkylamine of formula (IV) is 2,2-difluoroethyl-1-amine.

3. The Process according to claim 1, in which the compound of formula (II) is succinimide or phthalimide.

4. The Process according to claim 1, in which the compound of formula (II) is phthalimide.

5. The Process according to claim 1, in which the acid scavenger in (i) is a base chosen from tertiary amines, substituted or unsubstituted pyridines and substituted or unsubstituted quinolines, triethylamine, trimethylamine, diisopropylethylamine, tri-n-propylamine, tri-n-butylamine, tri-n-hexylamine, tricyclohexylamine, N-methylcyclohexylamine, N-methylpyrrolidine, N-methylpiperidine, N-ethylpiperidine, N,N-dimethylaniline, N-methylmorpholine, pyridine, 2-, 3- or 4-picoline, 2-methyl-5-ethylpyridine, 2,6-lutidine, 2,4,6-collidine, 4-dimethylaminopyridine, quinoline, quinaldine, N,N,N,N-tetramethyl-ethylenediamine, N,N-dimethyl-1,4-diazacyclohexane, N,N-diethyl-1,4-diazacyclohexane, 1,8-bis(dimethylamino)naphthalene, diazabicyclooctane (DABCO), diazabicyclononane (DBN), diazabicycloundecane (DBU), butylimidazole, methylimidazole, sodium hydroxide, potassium hydroxide, lithium hydroxide, calcium hydroxide, sodium carbonate, potassium carbonate, sodium hydrogencarbonate, potassium hydrogencarbonate, sodium acetate, KF and CsF.

6. The Process according to claim 1, in which the acid scavenger in (i) is a base which is diazabicycloundecane, potassium carbonate, sodium carbonate, KF or CsF.

7. The Process according to claim 5, in which the molar ratio of the base to the imide of formula (II) used is in a range of from 1:1 to 5:1.

8. The Process according to claim 1, in which inorganic acids are used in (ii).

9. The Process according to claim 8, in which the inorganic acid is hydrochloric acid, hydrobromic acid, sulphuric acid or phosphoric acid.

10. The Process according to claim 1, in which hydrazine hydrate is used in (ii).

11. The Process according to claim 1, in which the molar ratio of acid or hydrazine hydrate to the compound of the formula (III) is in a range of from 0.8:1 to 10:1.
